(19) United States
(12) Patent Application Publication
(10) Pub. No.: US 2017/0296766 A1
(43) Pub. Date: Oct. 19, 2017

(54) DEVICE FOR PRESSURE REGULATION OF A BREATHABLE GAS

(71) Applicant: NOMICS, Liege (BE)

(72) Inventors: Pierre ANSAY, Liege (BE); Bernard BECKERS, Liege (BE); Jean-Christian BOREL, Liege (BE); Jean-Louis PEPIN, Liege (BE)

(73) Assignee: NOMICS, Liege (BE)

(21) Appl. No.: 15/512,457

(22) PCT Filed: Sep. 18, 2015
(86) PCT No.: PCT/BE2015/000045

§ 371 Date: Mar. 17, 2017

(30) Foreign Application Priority Data
Sep. 19, 2014 (BE) BE2014/00714

(51) Int. Cl. A61M 16/00; A61M 16/06

(52) U.S. Cl. CPC A61M 16/0051; A61M 16/06; A61M 16/0069; A61M 2205/52; A61M 2205/3553; A61M 2205/3303; A61M 2205/15; A61M 2230/40; A61M 2230/63; A61M 2205/3331

(57) ABSTRACT

A breathable gas supply regulation device of gas supplied by a respiratory assistance apparatus in breath assistance comprising a first input receiving a leakage signal produced by a detection member. The device regulates the supply of breathable gas to the patient in response to a received leakage signal, comprises a second input receiving a patient mouth movement measurement signal. The device comprises a processing unit provided for generating a synchronisation signal between the movement signal and the leakage signal. The processing unit generates a modification amount indicating the modification to be applied to the supply of breathable gas.

The present invention relates to a breathable gas supply regulation device, said gas being supplied by a respiratory assistance apparatus in respiratory assistance to a patient, which device comprises a first input provided for receiving a leakage signal produced, in case of gas leakage detection, by a detection member provided to detect a leakage in the gas supplied by the respiratory assistance apparatus, which device is provided for regulating the supply of breathable gas to the patient in response to the received leakage signal.

Such a control device is known from the international patent application WO 2010/088543. The device is generally used in a respiratory assistance apparatus, provided for supplying breathable gas in breathing assistance to a patient who suffers from breathing insufficiency or from breathing troubles during sleep. The apparatus uses an interface, generally constituted by a mask, which is put on the patient's face. The regulation device of which the known apparatus is equipped, receives a leakage signal indicating that a leakage of the supplied gas has occurred, for example due to a displacement of the interface. The regulation device is provided for regulating the breathable gas supply to the patient in response to the received leakage signal. Thus the regulation device can maintain the pressure of the supplied gas when a leakage has been established.

A disadvantage of the known regulation device is that the origin of the leakage is not sufficiently known, in particular when the leakage is caused by the patient himself, for example when the leakage is the consequence of a patient's mouth movement. The known device is indeed not able to distinguish if the leakage is for example due to a displacement of the interface or to a patient's mouth movement. This can than lead to a regulation which is not always appropriate.

The object of the invention is to realise a breathable gas supply regulation device which is able to better recognise the origin of the leak and thus to better regulate the gas supply in case of a leakage.

To this purpose a regulation device according to the invention is characterised in that the device comprises a second input provided for receiving a patient mouth movement measurement signal produced by a mouth movement measurement device, which regulation device comprises a processing unit provided for generating a synchronisation signal by synchronising in time the movement measurement signal and the leakage signal and for establishing, under control of the synchronisation signal, if a variation of the value of the mouth movement measurement signal occurred and for generating a first control signal when establishing that a variation of value of the patient's mouth movement measurement signal occurred, which processing unit is provided for generating, under control of the first control signal, a modification amount indicating the modification to be applied to the supply of breathable gas supplied by the respiratory assistance apparatus. The mouth opening variation measurement signal enables to determine stereotyped movements of the mouth indicating for example the presence of an apnea or another breathing trouble during sleep or the awakening of the patient. By analysing if the leakage signal coincides in time with a variation of the mouth opening distance or with a stereotyped mouth movement, it is possible to establish that there is a link between the occurrence of the leakage and the mouth movement. The production of the first control signal, which takes place when the processing unit has established said coincidence in time, enables also to modify the breathable gas supply in function of the knowledge that the leakage was caused for example by a breathing trouble during sleep or by an apnea of the patient.

A first preferred embodiment of a device according to the invention is characterised in that the processing unit is provided for verifying if the variation of the value of the mouth movement measurement signal presents an oscillation in time, and for incorporating an increase of the gas supply in the modification amount of the gas supply when establishing that the value of the movement measurement signal oscillates in time. The presence of an oscillation in time in the distance variation measurement signal indicates that the patient must do efforts to breath and that an increase of the breathing channel resistance has occurred with the patient. If this is the case, an increase of the gas supply is required.

A second preferred embodiment of the device according to the invention is characterised in that it comprises a memory provided for temporarily storing therein the synchronisation signals and the movement measurement signals. This enables to store the leakage and the distance measurement signals for enabling in such a manner a later analysis.

Preferably the processing unit is provided for verifying if, after having generated the control signal, the value of the measurement signal of the movement variations remains essentially stable in time and for generating a second control signal when it has been established that the value of the measurement signal of the movement variations remains essentially stable in time, which processing unit is provided for incorporating in the modification amount a decrease of the gas supply under control of the second control signal. This enables to establish that the leakage is due for example to a change of the patient's position and thus that a reduction of the gas supply is preferred.

The invention also relates to a respiratory assistance apparatus comprising a regulation device according to the invention.

The invention will now be described in more details with reference to the drawings illustrating an embodiment of the regulation device and of the respiratory assistance apparatus according to the invention. In the drawings:

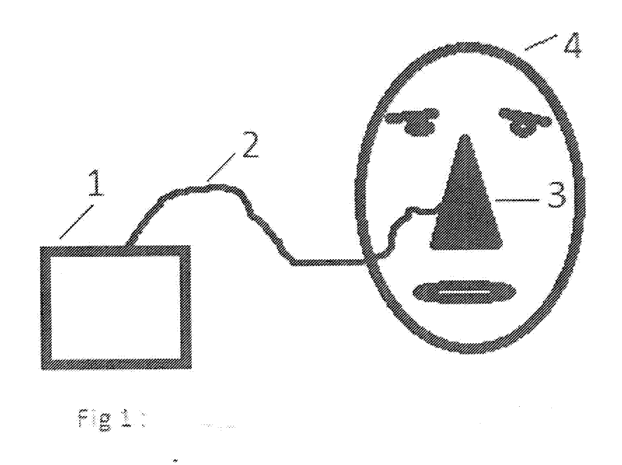

FIG. 1 shows a patient coupled to a respiratory assistance apparatus;

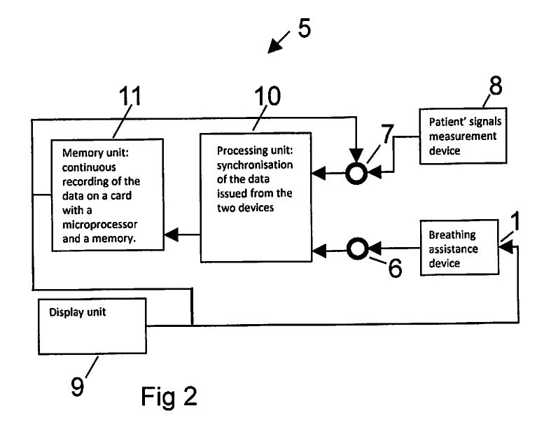

FIG. 2 illustrates schematically the regulation device according to the invention;

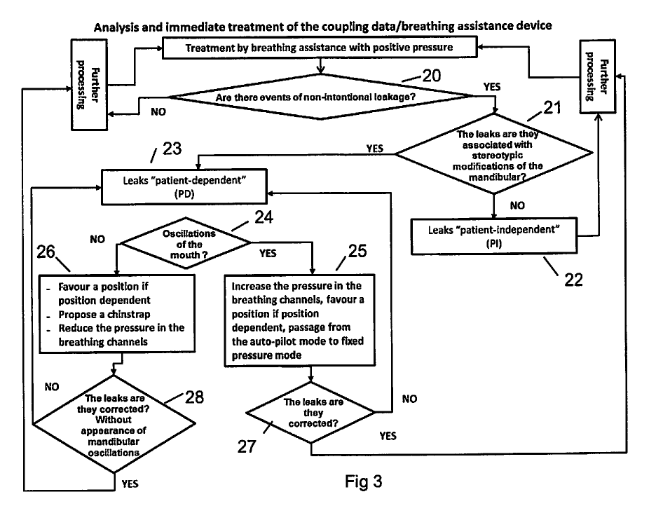

FIG. 3 illustrates by means of a flowchart a first embodiment of the functioning of the regulation device according to the invention;

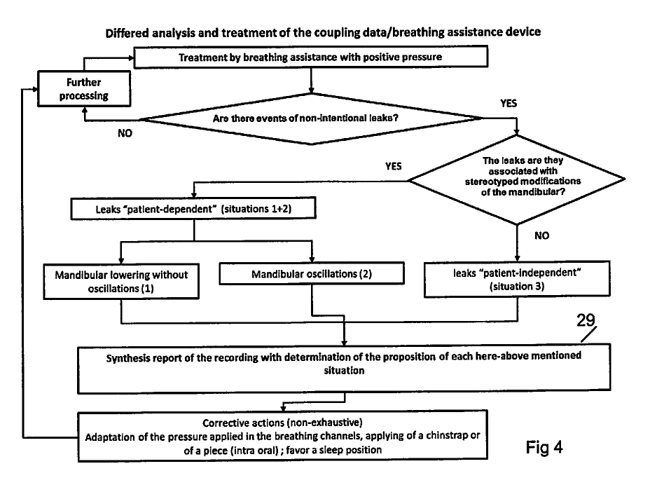

FIG. 4 illustrates by means of a flowchart a second embodiment of the functioning of the regulation device according to the invention;

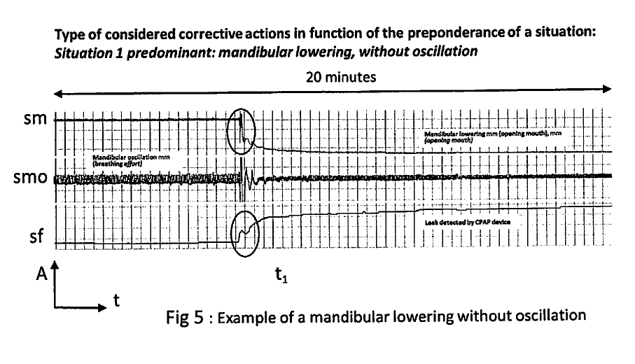

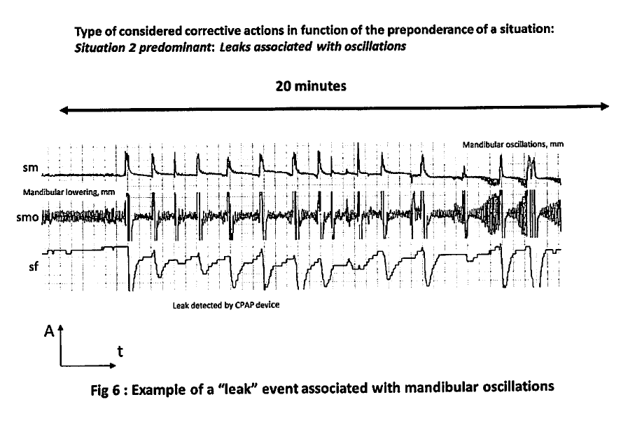

FIGS. 5 and 6 illustrate examples of leakage signals and distance measurements signals supplied to the regulation device according to the invention. In the drawings a same reference sign has been allotted to a same or analogous element.

FIG. 1 shows a patient 4 coupled to a respiratory assistance apparatus 1. The patient is coupled to the apparatus by means of an interface 3 connected to the apparatus by means of a supply tube 2. The respiratory assistance apparatus supplies breathable gas in breathing assistance to the patient. Those apparatuses are often called by the term CPAP (Continuous Positive Airway Pressure) or Bilevel Positive Airway Pressure (B-PAP) and are used for treating apnea syndromes during sleep or certain types of breathing insufficiency. The apparatus is provided for supplying breathable gas under pressure in the upper air channels of the patient. The interface 3 between the patient and the apparatus is generally formed by a mask put on the face, in particular on the nose and/or the mouth, of the patient.

The respiratory assistance apparatus is provided with on board software which records, when in use, a number of signals at each breathing cycle of the patient, such as the breathing flux and the pressure in the interface. In particular, the assistance apparatus is equipped with a leakage detection member (not shown in the drawing) for detecting a leakage in the breathable gas supplied to the patient. This leakage detection member will produce a leakage signal upon detection of a leakage at the level of the gas supply.

The respiratory assistance by positive pressure enables to maintain the permeability of the breathing channels of the patient during sleep and/or to reduce the breathing work and/or correct an instability of the breathing. If the interface is not correctly adapted, a leakage may occur. The type of leakage is “patient independent”, that is to say independent of the mouth movements. A second origin of those leakages is said of “patient-dependent” origin, as it is associated to the patient and not to his interface. The positioning of the patient or of his head can cause a leakage, an opening of the mouth enabling the gas to partly escape or a specific movement of the mouth can also cause a leakage. Knowing the origin of the leakages means enabling a better adaptation of the therapy and an improvement of the monitoring.

FIG. 2 illustrates schematically the regulation device according to the invention. This control device is either integrated in the assistance apparatus, or is formed by a separate unit, which can be connected to the respiratory assistance apparatus. The device 5 comprises a first input 6, provided for receiving a leakage signal produced by the leakage detection member of the breathing assistance apparatus 1. The device also comprises a second input 7 provided for receiving a patient mouth movement measurement signal. This movement measurement signal is generated by a patient mouth movement measurement device 8. Such a device is for example described in the patent EP 1 716 387. The description of the latter patent is incorporated by reference to the present description.

The first and the second input are connected to a processing unit 10, for example formed by a microprocessor. The processing unit is connected to the first and second input for receiving the leakage signal and the mouth movement measurement signal. The processing unit is provided for generating a synchronisation signal by synchronising in time the movement measurement signal and the leakage signal.

FIG. 5 illustrates a graphic with an example of a mouth movement measurement signal sm and a leakage signal sf. The horizontal axis of the graphic indicates the time and the vertical axis indicates the amplitude of the signal. The signal sm is a signal indicating a downward movement of the patient's mouth without however that an oscillation takes place. One sees indeed a drop down in the signal at the time t1, which indicates that a mouth movement took place, in particular that the patient's mouth went open. Practically at the same time t1, one will see that the amplitude of the signal sf has changed, which indicates that a leakage of the gas supplied to the patient occurred. One can thus establish that there exists a correlation in time between the mouth's movement and the occurrence of the leakage in the supply of the breathable gas to the patient.

FIG. 5 also shows a signal smo which represents an oscillation of the patient's mandibular, which is another way to express the patient's mouth movements. In this mouth movement measurement signal one will also see that around the moment t1 the amplitude of the signal changes.

FIG. 6 also shows the signals sm,smo and sf. The illustrated signals in FIG. 6 distinguish over the one of FIG. 5 by the presence of several oscillations in the signal. The oscillations in the signals sm and smo indicate that the patient's mouth is particularly instable and oscillates between an open state and a closed state. The signals sm and smo indicate an apnea state with the patient who is doing considerable efforts to breath. By doing a correlation in time between the signals sm or smo and sf, one will establish that the signal sf is also considerably instable, which indicates that there is an oscillation in the leakage detected by the leakage detection member. FIG. 6 also shows that there is a coincidence in time between the detection of a leakage and the mouth movement and the leakages vary according to a frequency comparable to the one at which the mouth varies.

When the leakage is not linked to a mouth movement, but to a bad positioning of the interface on the patient, the signals sm and smo remain substantially stable and when the interface is correctly repositioned, the amplitude of the leakage signal sf drops.

The present invention is based on this observation that there exists a correlation in time between the mouth movement and the occurrence of a leak in the gas supplied to the patient. It is the reason why the processing unit 10 is provided for synchronising in time the mouth movement signal and the leakage signal. It is this correlation which will enable to establish that the origin of the leakage is due to a mouth movement.

As mentioned here before, the processing unit is provided for generating the synchronisation signal. Because the leakage signal is only produced when a leakage occurs, the fact of synchronising in time the leakage signal with the mouth movement measuring signal will enable to establish if there exists a time link between the gas leak and the mouth movement.

The processing unit will produce (20) the synchronisation signal each time it receives the leakage signal. This is illustrated in step 20 shown in the flowchart illustrated in FIG. 3 and which illustrates a first embodiment of how the device according to the invention functions. The synchronisation signal is for example produced by coupling the leakage signal with the mouth movement measurement signal. The data processing unit is also provided for establishing (21), under control of the synchronisation signal, if a variation of the value of the mouth movement measurement signal (sm,smo) has occurred. When the processing unit establishes that the movement measurement signal does not comprise a variation (21,N) it signifies that the leakage is not caused by a mouth movement. In the latter case the processing unit cancels (22) the synchronisation signal.

On the contrary, if under control of the synchronisation signal it is established (21,Y) that the mouth movement measurement signal has varied, because the patient's mouth was opened (FIG. 5 or 6), the processing unit will produce (23) a first control signal. The latter will on his turn start the generation of a modification amount indicating the modification to be applied to the supply of breathable gas which has to be modified. This modification is for example realised by modifying the pressure under which the gas is supplied, or by modifying the control of the gas supply frequency. By gas supply frequency one understands the frequency at which a predetermined amount of gas is supplied to the patient. By stereotyped mouth movement it is for example meant a movement which characterises the presence of apnea or another trouble of breathing while sleeping or a movement characterising the awakening of the patient.

Preferably the amount at which the gas supply is modified is determined in function of the value of the mouth movement measurement signal. Thus during step 24 the processing unit will verify if the variation of the value of the movement measurement signal exceeds a predetermined threshold or represents a stereotyped mouth movement. If this is not the case the first control signal is not produced (26) and no modification is applied to the gas supply. Indeed, it is possible that the mouth movement measurement device establishes only a weak mouth movement which only cause a weak leakage, and that the leakage has already disappeared. In this case it is not necessary to modify the gas supply.

The value of the movement measurement signal is preferably taken into account upon determining the modification of the gas supply. Indeed, as illustrated in the FIGS. 6 and 5 the mouth movement can oscillate or not. To this purpose the processing unit will verify (24) if the variation of the value of the movement measurement signal presents an oscillation in time. This is for example realised by monitoring if, after the generation of a synchronisation signal, other synchronisation signals are produced. Indeed, as illustrated in FIG. 6, if the mouth oscillates, the leakage signal will also oscillate. On the contrary if the mouth does not oscillate, as illustrated in FIG. 5, the leakage signal remains stable. This analysis will thus enable to establish if the leakage is caused by an oscillation of the patient's mouth.

If the movement measurement signal oscillates (24,O), the modification amount will incorporate an increase (25) of the gas supply. If on the other hand the measurement signal remains essentially stable (26) in time, the processing unit will produce a second control signal. Under control of the second control signal the processing unit will adapt the modification amount of the gas supply, for example by incorporating therein a reduction of the gas pressure in order to reduce the leakage.

After having modified the gas supply to the patient the processing unit will verify (27) if the leakage is compensated. If this is not the case the process is taken up again as from step (24).

The device according to the invention is preferably equipped with a memory (11) connected to the processing unit (10) and provided for temporarily storing therein the synchronisation signals and the movement measurement signals. Eventually the device is connected to a display unit (9) which enables to display signals stored in the memory.

According to another embodiment illustrated in FIG. 4, the processing unit is provided for supplying (28) a synthesis report resuming the recording with determination of the proportion of each situation. This report also includes the leakage signals and the mouth movement measurement signals and suggest in function of those signals, actions to be considered by medical staff. This synthesis report is, eventually, available on a remote display or can be mailed by a communication network to medical staff.

1. A breathable gas supply regulation device, said gas being supplied by a respiratory assistance apparatus in respiratory assistance to a patient, which device comprises a first input provided for receiving a leakage signal produced, in case of a gas leakage detection, by a detection member provided to detect a leakage in the gas supplied by the respiratory assistance apparatus, which device is provided for regulating the supply of breathable gas to the patient in response to a received leakage signal, wherein the device comprises a second input provided for receiving a patient mouth movement measurement signal produced by a mouth movement measurement device, which regulation device comprises a processing unit provided for generating a synchronisation signal by synchronising in time the movement measurement signal and the leakage signal and for establishing, under control of the synchronisation signal, if a variation of the value of the mouth movement measurement signal occurred and for generating a first control signal when establishing that a variation of value of the mouth movement measurement signal occurred, which processing unit is provided for generating under control of the first control signal a modification amount indicating the modification to be applied to the supply of breathable gas supplied by the respiratory assistance apparatus.

2. The regulation device as claimed in claim 1, wherein the processing unit is provided for verifying if the variation of the value of the mouth movement measurement signal exceeds a predetermined threshold or represents a stereotyped mouth movement, and for generating the first control signal if the measurement signal exceeds the predetermined threshold or represents a stereotyped mouth movement.

3. The regulation device as claimed in claim 1, wherein the processing unit is provided for determining the modification amount also in function of the value of the movement measurement signal.

4. The regulation device as claimed in claim 1, wherein the processing unit is provided for verifying if the variation of the value of the mouth movement measurement signal presents an oscillation in time, and for incorporating an increase of the gas supply in the modification amount of the gas supply when establishing that the value of the movement measurement signal oscillates in time.

5. The regulation device as claimed in claim 1, wherein said device comprises a memory provided for temporarily storing therein the synchronisation signals and the movement measurement signals.

6. The regulation device as claimed in claim 1, wherein the processing unit is provided for verifying if, after having generated the control signal, the value of the measurement signal of the movement variations remains essentially stable in time and for generating a second control signal when it has been established that the value of the measurement signal of the movement variations remains essentially stable in time, which processing unit is provided for adapting the modification amount of the gas supply under control of the second control signal.

7. The regulation device as claimed in claim 1, wherein said device is provided for regulating the gas supply by a pressure control of the supplied gas.

8. The regulation device as claimed in claim 1, wherein said device is provided for regulating the gas supply by a pressure control at inhalation and at exhalation of the supplied gas.

9. The regulation device as claimed in claim 1, wherein said device is provided for regulating the gas supply by a control of the frequency of the supplied gas.

10. Respiratory assistance apparatus comprising a regulation device as claimed in claim 1.
